(19) United States
(12) Patent Application Publication
(10) Pub. No.: US 2016/0120802 A1
(43) Pub. Date: May 5, 2016

(54) ORALLY-DISINTEGRATING OSELTAMIVIR TABLET AND METHOD FOR PREPARING THE SAME

(71) Applicant: Zhongshuai Pharmaceutical Sci & Tech Incorporated Co., Ltd., Zhengzhou (CN)

(72) Inventors: Weidong SUN, Zhengzhou (CN); Fengxiao REN, Zhengzhou (CN)

(21) Appl. No.: 14/992,040

(22) Filed: Jan. 11, 2016

Related U.S. Application Data
(63) Continuation-in-part of application No. PCT/CN2014/000835, filed on Sep. 10, 2014.

(30) Foreign Application Priority Data
Jul. 11, 2013 (CN) 201310290328.7

(51) Int. Cl. A61K 9/00; A61K 9/20; A61K 31/215

(52) U.S. Cl. CPC A61K 9/0056; A61K 31/215; A61K 9/2004

(57) ABSTRACT

An orally-disintegrating oseltamivir tablet, including: between 10 and 50 wt. % of a taste-masking pellet, between 30 and 80 wt. % of a first filler, between 1 and 6 wt. % of a first adhesive, between 2 and 10 wt. % of a disintegrant, between 0 and 5 wt. % of a flavoring agent, between 0.5 and 2.5 wt. % of a lubricant. The taste-masking pellet includes: an active ingredient-loaded pellet core and a coating layer. The active ingredient of the pellet core is oseltamivir or a pharmaceutically acceptable salt of oseltamivir and accounts for between 10 and 40 wt. % of a total weight of the taste-masking pellet. The coating layer include a polyacrylic acid resin IV and accounts for between 1 and 50 wt. % of the total weight of the taste-masking pellet. The diameter of the taste-masking pellet is between 0.10 and 0.50 mm.

### CROSS-REFERENCE TO RELATED APPLICATIONS

This application is a continuation-in-part of International Patent Application No. PCT/CN2014/000835 with an international filing date of Sep. 10, 2014, designating the United States, now pending, and further claims priority benefits to Chinese Patent Application No. 201310290328.7 filed Jul. 11, 2013. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

1. Field of the Invention

The invention relates to an orally-disintegrating oseltamivir tablet and a method for preparing the same.

2. Description of the Related Art

Oseltamivir, having the chemical name of (3R,4R,5S)-4-acetamido-5-amino-3(1-ethylpropoxy)-1-cyclohexene-1-carboxylate and the structural formula (I), can act on neuraminidase (NA), to inhibit the replication and spreading of the viruses.

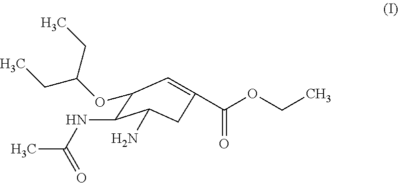 [SMILES: CCOC(=O)C1=CC(OC(CC)CC)C(NC(C)=O)C(N)C1]

Because oseltamivir has strong bitter taste and the commercial oseltamivir adopts capsules for the dosage form, it is difficult for children and infants to swallow the oseltamivir capsule. To cover the bitter taste of oseltamivir, various encapsulation techniques have been tried. However, based on conventional encapsulation techniques, the active ingredient cannot be completely encapsulated, or the pill has too large a particle size thus affecting the administration, or the pill has too small a particle size thus leading to low load efficiency.

### SUMMARY OF THE INVENTION

In view of the above-described problems, it is one objective of the invention to provide an orally-disintegrating oseltamivir tablet. The orally-disintegrating tablet of the invention features fast dissolution, short disintegration time, good taste, and no gritty irritation to the oral cavity, thus being suitable for administration by children and particularly infants for preventing and treating influenza of children and particularly infants.

Unless otherwise indicated, oseltamivir, as used herein, refers to oseltamivir phosphate.

It is another objective of the invention to provide a method for preparing the orally-disintegrating oseltamivir tablet. The preparation method is able to meet the demands of the large scale industrialized production. The hardness of the prepared tablet exceeds 40 N, which facilitates the package and the transportation. In addition, the method has simple operation, stable products, high reproducibility, and is prone to large scale production.

To achieve the above objective, in accordance with one embodiment of the invention, there is provided an orally-disintegrating oseltamivir tablet. The tablet comprises: between 10 and 50 wt. % of a taste-masking pellet, between 30 and 80 wt. % of a first filler, between 1 and 6 wt. % of a first adhesive, between 2 and 10 wt. % of a disintegrant, between 0 and 5 wt. % of a flavoring agent, between 0.5 and 2.5 wt. % of a lubricant.

The taste-masking pellet comprises: an active ingredient-loaded pellet core and a coating layer. The active ingredient of the pellet core is oseltamivir having a formula I

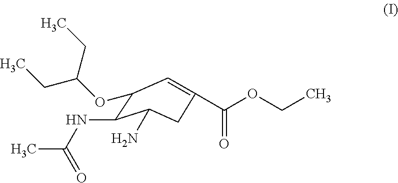 [SMILES: CCOC(=O)C1=CC(OC(CC)CC)C(NC(C)=O)C(N)C1]

or a pharmaceutically acceptable salt of oseltamivir, and accounts for between 10 and 40 wt. % of a total weight of the taste-masking pellet. The coating layer comprises a polyacrylic acid resin IV and accounts for between 1 and 50 wt. % of the total weight of the taste-masking pellet, preferably, the coating layer accounts for between 5 and 50 wt. % of the total weight of the taste-masking pellet. A diameter of the taste-masking pellet is between 0.10 and 0.50 mm, preferably between 0.15 and 0.35 mm. The salt of oseltamivir is oseltamivir phosphate.

In a class of this embodiment, the pellet core of the taste-masking pellet is prepared by a blank pellet, the drug, a second filler, a second adhesive, and an anti-sticking agent. The weight percentage amounts of the components in the pellet are as follows: between 10 and 40 wt. % of the drug, between 20 and 60 wt. % of the blank pellet, between 0 and 50 wt. % of the second filler, between 1 and 20 wt. % of the second adhesive, and between 0.5 and 5 wt. % of the anti-sticking agent. The blank pellet is at least one selected from the group consisting of a sucrose pellet, a microcrystalline cellulose pellet, a starch pellet, a lactose-microcrystalline cellulose pellet, a starch-microcrystalline cellulose pellet, and a sucrose-starch pellet. The second filler is at least one selected from the group consisting of sucrose, lactose, mannitol, a starch, a microcrystalline cellulose, an algal polysaccharide, a chitosan. The second adhesive is at least one selected from the group consisting of water, ethanol, a hydroxypropyl methyl cellulose, a polyacrylic acid resin, a hydroxypropyl cellulose, a povidone, a polyvinyl alcohol, and a carboxymethylcellulose sodium. The anti-sticking agent is at least one selected from the group consisting of a talc, colloidal silica, magnesium stearate, calcium stearate, magnesium silicate, and glyceryl monostearate.

In a class of this embodiment, in the taste-masking pellet, the blank pellet is the sucrose pellet, the second filler is lactose and/or mannitol, the second adhesive is the hydroxypropyl methyl cellulose, and the anti-sticking agent is the talc. The weight percentages of the components in the taste-masking pellet are as follows: between 10 and 30 wt. % of oseltamivir, between 5 and 50 wt. % of the polyacrylic acid resin IV, between 20 and 60 wt. % of the sucrose pellet, between 0 and 50 wt. % of lactose and/or mannitol, between 1 and 20 wt. % of the hydroxypropyl methyl cellulose, and between 0.5 and 50 wt. % of the talc.

In a class of this embodiment, the weight percentage amounts of the components in taste-masking pellet are as follows: 23.8 wt. % of oseltamivir, 16.7 wt. % of the polyacrylic acid resin IV, 49.6 wt. % of the sucrose pellet, 7.9 wt. % of the hydroxypropyl methyl cellulose, and 2.0 wt. % of the talc.

A method for preparing the taste-masking pellet comprises:
1) grinding a raw material and sieving the ground raw material by a 120-mesh sieve, grinding an excipient and sieving the excipient by a 80-mesh sieve, adding oseltamivir, the filler, and the anti-sticking agent to an adhesive solution while stiffing to form a suspension comprising materials for a drug-loaded layer. Preferably, the drug-layering process of the suspension is performed in a fluidized bed or a coating pan. The materials for the drug-loaded layer is atomized at a proper pressure, sprayed into the fluidized bed, so that the materials for the drug-loaded layer are loaded on the blank pellets and form the drug layer thereon at a certain temperature. Under a certain fluidized velocity, the blank pellets are transformed into active ingredient-loaded pellet cores having uniform contents and particle sizes. According to the originally adopted particle size of the blank pellets and the corresponding formulation and technology, the diameter of the active ingredient-loaded pellet cores can be controlled within a range of between 0.10 and 0.50 mm, and preferably within a range of 0.25±0.10
2) adding the polyacrylic acid resin IV to an ethanol solution to yield a polyacrylic acid resin IV solution, performing a coating process in the fluidized bed or the coating pan, atomizing the polyacrylic acid resin IV solution at a proper pressure and spraying the polyacrylic acid resin IV solution into the fluidized bed to allow coating materials to adhere to the active ingredient-loaded pellet cores at a certain temperature.

In a class of this embodiment, the adhesive solution is prepared by mixing an adhesive with water, and the weight of the water is between 5 and 100 times of the weight of the adhesive. The coating solution is prepared by mixing the polyacrylic acid resin IV with the ethanol solution. The ethanol solution comprises between 50 and 99 wt. % of ethanol. The weight of ethanol is between 5 and 100 of the weight of the polyacrylic acid resin IV.

In a class of this embodiment, the prepared taste-masking pellet is adopted as the main drug to be mixed with the first filler, the first adhesive, the flavoring agent, the disintegrant, and the lubricant to prepare the orally-disintegrating oseltamivir tablet using the common pharmaceutics techniques. The first filler is at least one selected from the group consisting of mannitol, xylitol, sucrose, fructose, glucose, maltose, aminoacetic acid, sorbitol, a microcrystalline cellulose, and lactose. The first adhesive is at least one selected from the group consisting of a hydroxypropyl methyl cellulose, a polyacrylic acid resin, a hydroxypropyl cellulose, a povidone, a polyvinyl alcohol, and a carboxymethylcellulose sodium. The disintegrant is at least one selected from a sodium carboxyl methyl starch, a crospovidone, a croscarmellose sodium, a substituted hydroxypropyl cellulose, and a carboxymethylcellulose calcium. The flavoring agent is at least one selected from aspartame, Acesulfame-K, saccharin sodium, a dextran, a stevioside, citric acid, an essence, and a perfume. The lubricant is at least one selected from the group consisting of a talc, a hydrogenated vegetable oil, sodium stearyl fumarate, magnesium stearate, stearyl alcohol.

In a class of this embodiment, the first filler is mannitol and/or the microcrystalline cellulose. The first adhesive is the povidone; the disintegrant is the crospovidone and/or the substituted hydroxypropyl cellulose. The flavoring agent is aspartame and/or citric acid and/or a strawberry essence. The lubricant is sodium stearyl fumarate and/or magnesium stearate.

In a class of this embodiment, the percentage amounts of the components in the tablet are as follows: between 10 and 50 wt. % of the taste-masking pellet having the diameter of between 0.15 and 0.35 mm, between 40 and 80 wt. % of mannitol and/or the microcrystalline cellulose, between 1 and 3 wt. % of the povidone, between 5 and 10 wt. % of the crospovidone and/or the substituted hydroxypropyl cellulose, between 1 and 4 wt. % of aspartame and/or citric acid and/or the strawberry essence, and between 0.5 and 1.5 wt. % of sodium stearyl fumarate and/or magnesium stearate.

In a class of this embodiment, the percentage amounts of the components in the tablet are as follows: between 25 and 35 wt. % of the taste-masking pellet having the diameter of between 0.15 and 0.35 mm, between 40 and 50 wt. % of mannitol, between 5 and 8 wt. % of the microcrystalline cellulose, between 1 and 3 wt. % of the povidone, between 6 and 10 wt. % of the crospovidone, between 2 and 3 wt. % of aspartame, between 0.5 and 1 wt. % of citric acid, between 0.5 and 1 wt. % the strawberry essence, and between 1.0 and 1.5 wt. % of sodium stearyl fumarate.

In a class of this embodiment, a hardness of the tablet exceeds 40 N. The tablet is orally disintegrated within 30 seconds in the absence of bitter taste.

In accordance with another embodiment of the invention, there is provided a method for preparing the orally-disintegrating oseltamivir tablet. The method comprises:
a) grinding oseltamivir, and processing the ground oseltamivir into active ingredient-loaded pellet cores using a coating machine; adding the polyacrylic acid resin IV to an ethanol solution, and coating a resulting solution on the active ingredient-loaded pellet cores using a fluidized bed or a coating pan to form a taste-masking layer on the pellet cores, whereby yielding the taste-masking pellets; and
b) uniformly mixing the taste-masking pellets with the first filler, the first adhesive, the flavoring agent, the disintegrant, and the lubricant, and tableting a resulting mixture.

In a class of this embodiment, the tablet is scored into between 4 and 12 equal parts, and preferably between 6 and 12 equal parts. An effective dose of oseltamivir is between 5 and 50 mg, and preferably between 10 and 30 mg.

Advantages of the orally-disintegrating oseltamivir tablet and the preparation method thereof of the invention are further illustrated hereinbelow combining with experiment data.

Test 1 Measurement of Dissolution and Disintegration Time

Method of dissolution measurement: a sample of the orally-disintegrating tablet is collected. Based on the dissolution test method of oseltamivir capsules described in USP36, the apparatus required in the second method of the determination of dissolution is provided. 500 mL of aqueous hydrochloric acid solution (0.1 mol/L) is used as a releasing medium, the rotary speed is 50 rpm. At the 5, 10, and 20 minutes, 10 mL of the solution is collected and filtered using a 45 μm filter membrane, and 10 mL of the dissolution medium is instantly added. The subsequent filtrate is collected as the test solution. In the meanwhile, oseltamivir is accurately weighed as a control solution. Appropriate releasing medium is added, heated, and diluted to prepare the control solution, 1 mL of which contains 20 μg of active ingredient. The absorbance of the test solution and the control solution are measured as 240 nm using an ultraviolet-visible spectroscopy (The Supplemented Content IV A of Chinese Pharmacopoeia Edition 2010), to calculate the dissolution at different times.

Method of measurement of disintegration time: a sample of the orally-disintegrating tablet is collected, and based on the measurement method of disintegration time (Tablets Measurement Method, The Supplemented Content XA of Chinese Pharmacopoeia Edition 2010), a basket is suspended on a metal bracket, and immerged in a 1000 mL breaker. Adjust the position of the basket so that when the basket descends, the sieve mesh is 25 far from the bottom of the breaker. The breaker contains water having a temperature of 37±1° C. Adjust the water height so that when the basket ascends, the sieve mesh is 15 mm lower than the water level. Six pieces of samples are collected and placed in glass tubes in the basket. The disintegration tester is started, when the tablets encounter water, time is recorded. Calculate the time that the granules pass the sieve mesh completely.

The measurement results are listed in Table 1.

TABLE 1
Results of dissolution and disintegration
time of orally-disintegrating tablets

| | Disintegration | Dissolution | | |
|---|---|---|---|---|
| Samples | time (s) | 5 min (%) | 10 min (%) | 20 min (%) |
| Example 1 | 15 | 98.6 | 99.2 | 99.5 |
| Example 2 | 18 | 97.1 | 99.8 | 99.7 |
| Example 3 | 10 | 99.2 | 99.6 | 100.3 |
| Example 4 | 25 | 95.0 | 98.9 | 99.3 |
| Example 5 | 18 | 96.6 | 99.9 | 99.6 |
| Example 6 | 20 | 95.2 | 98.6 | 99.4 |
| Example 7 | 19 | 97.3 | 99.4 | 99.5 |

Based on the measurement results, the orally-disintegrating tablet of the invention has fast disintegration speed and high release rate, it can be disintegrated completely within 30 seconds, and dissolve out in 5 min. Therefore, the orally-disintegrating tablet can take effect rapidly, which conforms to the compliance of children and infants and is suitable for industrial production.

Test 2 Influence of the Composition of the Invention on Pigeons Vomiting Model

1. Experimental Animal: Healthy Pigeons (Both Males and Females were Operable), 350±50 g of Weights, Common Grades, and Purchased from a Breeding Base.

2. Experimental Drugs and Administration Doses:

70 healthy pigeons were randomly divided into 7 groups and adaptively fed with normal diets for 1 week before the experiment. Diets were prohibited within 4 hrs before the experiment, the room temperature was maintained at between 22 and 24° C., and the feeding environment was kept clean and well ventilated. The pigeons were administered with drugs according to the following groups, the administration dosage corresponded to 30 mg oseltamivir per kg of the pigeon's weight, or the equivalent amount of the starch. The indexes were closely observed, and normal diets were recovered 8 hrs after the drug administration.

Experimental groups 1-3, three groups, administered with orally-disintegrating tablets comprising oseltamivir prepared by Examples 1-3 with a dosage equivalent to 30 mg of oseltamivir per kg of the pigeon's weight.

Contrast group A, one group, administered with the raw material of oseltamivir with a dosage equivalent to 30 mg of oseltamivir per kg of the pigeon's weight.

Contrast group B, one group, administered with the polyacrylic acid resin IV with a dosage equivalent to 30 mg of the starch per kg of the pigeon's weight.

Contrast group C, one group, administered with a physical mixture of the raw material of oseltamivir and the excipients, and a dosage of the physical mixture was equivalent to 30 mg of oseltamivir per kg of the pigeon's weight.

Contrast group D, one group, administered with the commercial oseltamivir phosphate capsules manufactured by Roche with a dosage equivalent to 30 mg of oseltamivir per kg of the pigeon's weight.

3. Experimental Method

Observation indexes: latent periods of vomiting (the period from the administration to the first vomiting), vomiting times (occurrences of the vomiting after the drug administration, each occurrence of the vomiting refers to the duration from neck stretching, mouth opening, shrugging, and abdominal contraction of the pigeons to recovery of the quiet state of the pigeons), and vomiting frequencies (referring to the times of neck stretching, mouth opening, shrugging, and abdominal contraction of the pigeons).

The pigeons in each group were taken drugs by intragastric administration with a dosage equivalent to 30 mg of oseltamivir per kg of the pigeon's weight. The latent periods of vomiting (min) after the intragastric administration, as well as the vomiting times and the vomiting frequency in 5 hrs after the intragastric administration were recorded.

In each group, the number of the vomiting pigeons (n) was calculated. For the vomiting pigeons (including vomiting with vomitus and dry vomiting in the absence of vomitus), the latent period of vomiting (min, x̅±s), the average vomit times, and the average vomit frequencies were calculated, and results were listed in the following table 2.

TABLE 2
Comparison of vomiting phenomenon among experimental pigeons ( x̅ ± s, n = 10)

| | | | Average | | |
|---|---|---|---|---|---|
| | Number of | Latent | vomiting | | |
| | vomiting | period for | times | Vomiting | |
| | pigeons | vomiting | (within 5 | frequencies | |
| Groups | (n) | (min) | hrs) | (times) | Note |
| Example 1 | 2 | 168.1 ± 16.9 | 3.5 | 7.0 ± 1.4 | Dry vomiting |
| Example 2 | 1 | 177.3 ± 0 | 3.0 | 5.0 ± 0 | Dry vomiting |
| Example 3 | 2 | 171.2 ± 17.6 | 4.0 | 8.0 ± 2.8 | Dry vomiting |
| Contrast group A | 9 | 18.9 ± 5.4 | 11.1 | 15.8 ± 2.1 | Seven times of vomiting |
| | | | | | with vomitus, and the |
| | | | | | remaining were dry |
| | | | | | vomiting |
| Contrast group B | 1 | 220.9 ± 0 | 2.0 | 4.0 ± 0 | Dry vomiting |
| Contrast group C | 8 | 27.7 ± 7.8 | 9.8 | 13.4 ± 3.7 | Six times of vomiting |
| | | | | | with vomitus, and the |
| | | | | | remaining were dry |
| | | | | | vomiting |
| Contrast group D | 8 | 29.8 ± 9.6 | 9.4 | 12.5 ± 2.4 | Six times of vomiting |
| | | | | | with vomitus, and the |
| | | | | | remaining were dry |
| | | | | | vomiting |

It was known from the experiment results that the pigeons administered with the orally-disintegrating tablets comprising oseltamivir had low occurrence of vomiting, long latent periods of vomiting, and the vomiting times were few and the vomiting frequency was low.

Test 3 Optimized Formula Screening

1. The optimized formula was screened as follows:

1) Polyacrylic Acid Resin IV

Oseltamivir has strong bitter taste, and when oseltamivir is orally administered and directly contacts with the oral mucosa, the bitter taste can be obviously felt. Thus, to cover the bitter taste and reduce the irritation of oseltamivir, an isolation layer is coated on the active ingredient-loaded pellet cores. The methylcellulose, the polyacrylic acid resin IV, hydroxypropyl methyl cellulose, the hydroxypropyl cellulose, the povidone, the ethyl cellulose were compared, and the polyacrylic acid resin IV was finally selected as the coating material for taste-masking. The polyacrylic acid resin IV is the common coating material for taste-masking, herein different thickness of the polyacrylic acid resin IV layer, i. e., different proportions of the polyacrylic acid resin IV, were adopted to examine the influence thereof on the taste-masking effect.

A large batch of active ingredient-loaded pellet cores were prepared and processed by ethanol solutions comprising the polyacrylic acid resin IV prepared according to the same formula, so as to test the taste-masking effect of different amounts of the ethanol solutions comprising the polyacrylic acid resin IV. It was indicated from the test results that under the same circumstances, the taste-masking effect satisfied the requirements when the proportion of the polyacrylic acid resin IV as the coating layer was 16.7 wt. %. And when the proportion of the polyacrylic acid resin IV was 20 wt. %, the taste-masking effect was slightly better than that of the 16.7 wt. % of the polyacrylic acid resin IV, while in the condition of the 20 wt. % of the polyacrylic acid resin IV, the coating process consumed much time and labor and resulted in energy waste. Thus, the proportion of 16.7 wt. % of the polyacrylic acid resin IV was adopted as the optimized craft formula.

2) Blank Pellets

When adopting the fluidized bed or the coating pan for performing the drug-layering process, the drug contained suspension is required to be adhered to a carrier having a certain particle size. The most common blank carrier in the industrial production is blank small balls, i. e., the blank pellets, prepared using a certain pharmaceutical excipient. According to the difference of the pharmaceutical excipients, the blank pellets can be divided into the sucrose pellets, the microcrystalline cellulose pellets, the starch pellets, the lactose-microcrystalline cellulose pellets, the starch-microcrystalline cellulose pellets, and the sucrose-starch pellets. Of them, the sucrose pellets are the most common blank pellets and feature easy integration, low friability, small granularity deviation, high roundness, and narrow distribution range of the particle size. And it was indicated from the tests that the sucrose pellets satisfied the demands on the blank pellet. Thus, the sucrose pellets were selected as the blank pellets.

3) Adhesive

The addition of a certain amount of adhesive is required to adhere the main drug to the blank pellets. Hydroxypropyl methyl cellulose is white or yellowish powder, odorless, tasteless, stable to light, heat, and moisture, and is water soluble at any pH value at a temperature of less than 60° C. and a mixed solvent (1:1) of ethanol, propanol, or isopropanol having a concentration of 70 wt. % and methylene chloride, thereby being the most widely applied adhesive. Herein the hydroxypropyl methyl cellulose having the low viscosity grade (5 cPa·s) was adopted as the adhesive in the formula for preparing the active ingredient-loaded pellet cores.

Different amounts of the hydroxypropyl methyl cellulose were screened based on the same coating parameters. The recovery rate of the main drug in the active ingredient-loaded pellet cores obtained from the drug-layering process and the influence on the drug dissolution after the drug-layering process were employed as assessment indexes for the screening. It was indicated from the experiment results that under the same circumstance, when the proportion of the hydroxypropyl methyl cellulose as the adhesive was 7.9 wt. %, the recovery rate of the main drug in the active ingredient-loaded pellet cores reached 97. 8 wt. %, and such proportion had no influence on the dissolution of the main drug in the pellets.

4) Anti-Sticking Agent

In the drug-layering process and the coating process for preparing the oseltamivir pellets, the active ingredient-loaded pellet cores were easily adhered to each other and aggregated to form clusters. Thus, a certain amount of anti-sticking agent was required, herein the talc which satisfied the standard of the Chinese pharmacopoeia, 2010 edition, was adopted as the anti-sticking agent. The talc is white or off-white, and tiny powder in the absence of grittiness, and has an oily feel when being touched. The talc is odorless, tasteless, and is non-dissolvable in water, diluted acid, and diluted hydroxide alkalis. As the pharmaceutical excipient, the talc features wide range, non-toxicity, odorless, high whiteness, good compatibility, strong glossiness, soft tastes, and strong smooth degree. Besides, the talc has a pH value of between 7 and 9, and the original product characters do not change due to the decomposition thereof.

It was indicated from the experiment results that under the same circumstances, the addition of 2.0 wt. % of the talc was able to obviously improve the phenomenon of mutual adhesion and aggregation of the active ingredient-loaded pellet cores, thus satisfying the requirements.

2. A preferable formula of the orally-disintegrating tablet is summarized as follows.

1) The First Filler

The addition of the first filler increases the weight and volume of the orally-disintegrating tablet, which favors the shaping of the tablet. Acceptable first fillers comprise starch, sugar, cellulose, and inorganic salts. As white or colorless crystalline powders, mannitol has stable chemical properties and is freely soluble in water, soluble in glycerin, slightly soluble in ethanol. So, it is particularly suitable for being used as filler. The prepared tablet has smooth surface and beautiful appearance, the sweetness thereof is moderate. Microcrystalline cellulose has good fluidity and compressibility, and strong bonding force, so that the resulting tablet exhibits appropriate hardness, and has functions of bonding, flow-aiding, and disintegration. Single mannitol as the filler often causes the tablet has insufficient compressibility and low hardness. In this invention, appropriate microcrystalline cellulose is added to improve the compressibility and hardness of the tablet.

Experimental results show, when 40-50 wt. % of mannitol and 5-8 wt. % of microcrystalline cellulose are added as the first filler, the orally-disintegrating tablet exhibits high hardness and good taste.

2) The First Adhesive

Because the excipients of the orally-disintegrating tablet lack of viscosity, a sticky material is needed to shape the tablet. The sticky material is called first adhesive. The orally-disintegrating tablet is expected to be disintegrated within 30 seconds in the mouth, so the first adhesive is supposed to have good taste and water solubility. Povidone, which is a water-soluble, white or milky white power, and has high adhesive properties, is a common adhesive for solid preparations. Experimental results show, when 1-3 wt. % of povidone is added as the first adhesive, the orally-disintegrating tablet exhibits high hardness, good friability and good taste.

3) The Disintegrant

To ensure the orally-disintegrating tablet to be disintegrated within 30 seconds in the mouth, a disintegrant is a must. The disintegrant operates to eliminate the bonding force of the adhesive and the mechanical pressure resulting from the tablet pressing, so as to promote the solubility of the active ingredient, thus increasing the dissolution rate of the active ingredient.

Crospovidone is a white solid and has strong hydratability (HK: 5.6) and water swelling capacity, so it is often used as a disintegrant for water-insoluble tablets. Experimental results show, when 6-10 wt. % of cropovidone is added as the disintegrant, the disintegration speed of the orally-disintegrating tablet is significantly improved, specifically, the time is no more than 30 seconds in the mouth.

4) The Flavoring Agent

To ensure the compliance for the patients, the orally-disintegrating tablet is supposed to have good color, aroma and taste. Aspartame, citric acid and essence are common flavoring agent. Aspartame has high sweetness and low heat quantity, is a safe sweetening agent. Citric acid has mild and refreshing acidity, and can enhance appetite and promote the absorption of calcium phosphorus in the body. Experimental results show, when 2-3 wt. % of aspartame, 0.5-1 wt. % of citric acid, and 0.5-1 wt. % of strawberry essence are added as the flavoring agent, the orally-disintegrating tablet exhibits good taste.

5) The Lubricant

The lubricant operates to reduce the frictional force between granules or tablets and the punch pin and punch die in the course of tableting, so as to increase the lubricity of the granules and ensure the good filling and uniform density. Sodium stearyl fumarate is a nontoxic and nonirritating white power, and is a common hydrophilic lubricant.

Experimental results show, when 1.0-1.5 wt. % of sodium stearyl fumarate is added as the lubricant, the frictional force between the tablets and the punch pin and punch die in the course of tableting is significantly reduced.

Advantages of the orally-disintegrating oseltamivir tablet according to embodiments of the invention are summarized as follows.

1. The orally-disintegrating oseltamivir tablet prepared in the invention has no toxicity or side effects, and thus being convenient for long term treatment of influenza of children and infants and improving the medication compliance. The orally-disintegrating tablet is odorless and tasteless and has good roundness and no gritty irritation to the oral cavity. The dosage form and specifications of the orally-disintegrating tablet are specifically designed for children and infants, thus preventing the waste of the active ingredient. In addition, the tablet comprises multiple-scaled markers, which can ensure the accurate dosage amount.

2. Due to the introduction of the taste-masking pellet and flavoring agent, the bitterness of the orally-disintegrating tablet is greatly softened, and the tablet has good taste. Both the excipient and the preparation of the orally-disintegrating tablet of the invention are accessible, feasible, and suitable for large scale of industrial production. The preparation method of the tablet has excellent repeatability. Since the pellets of different specification have the same formula and craft, the pellets of different specifications can be obtained as long as changing the filling amount of the pellets. Particularly, the preferred formula and preparation method of the invention are both the best screened technical solutions. The pellets, prepared based on the optimized formula and the active ingredient-layering process in the fluidized bed, are odorless and tasteless and are able to realize the good release performance of the formulation thereof in vivo. Pellets of different specifications can be prepared to satisfy the requirements of children and infants with different weights.

3. The orally-disintegrating tablet of the invention have an effective dose of between 5 and 50 mg and have different specifications corresponding to administration objects of different weights. It is known from the in vivo experiments that when the administration level is between 1.0 and 4.0 mg/kg, the oseltamivir pellets of different specifications have the same in vivo pharmacokinetic parameters, thus satisfying the requirements on the blood concentration in therapy. Experiments show that, the orally-disintegrating tablet of the invention has short disintegration time, quick dissolution speed, and good taste, which can better meet the requirements of children and infants. In addition, the orally-disintegrating tablet has the same release rate as the capsules on the market. The in vivo pharmacokinetics shows, the tablet has the bioavailability as the capsules on the market.

4. The orally-disintegrating tablet has simple and feasible preparation process. The active ingredient-layering and the tableting processes can be adopted, and the yield thereof reaches 90 wt. %, which satisfies the requirements on the large-scale production. In the laboratory production, between 10000 and 30000 units of magnification production have been accomplished, and the production efficiency is high. Specifications of 5-50 mg of the orally-disintegrating tablet can be prepared. In the meanwhile, the orally-disintegrating tablet can be disintegrated within 30 seconds, and the hardness thereof can reach more than 40 N, which favors the packing, transportation and carrying for patients.

5. It is indicated from the accelerated stability test that the orally-disintegrating tablet has stable characters within 12 months, and the active ingredient content and related substances are all within a controllable range, thus the orally-disintegrating tablet is suitable for industrialized production.

### DETAILED DESCRIPTION OF THE EMBODIMENTS

For further illustrating the invention, experiments detailing an orally-disintegrating oseltamivir tablet and a method for preparing the same are described below. It should be noted that the following examples are intended to describe and not to limit the invention.

### Example 1

### Orally-Disintegrating Oseltamivir Tablet and Preparation Method Thereof

Taste-masking pellets comprising oseltamivir were first prepared, mixed with other excipients according to a formula, and then tableted into tablets, so as to form the orally-disintegrating tablets comprising oseltamivir.

1) Preparation Process of the Taste-Masking Pellets Comprising Oseltamivir

The raw material of oseltamivir and excipients were ground and screened by a 100-mesh sieve. 150.0 g of a starch and 150.0 g of oseltamivir were then put into a centrifugal coating pan. A temperature in the centrifugal coating pan was regulated to be 45° C., and an air supply rate therein was regulated to be 65 m³*h⁻¹. 300 g of a 75 wt. % ethanol solution as a second adhesive solution was sprayed by a peristaltic pump into the centrifugal coating pan at a flow rate of 3 mL/min for performing a drug-layering process, during which, an atomization pressure was controlled at 1.0 bar, and a solution supply rate was gradually increased to 6 mL/min until the second adhesive solution was consumed. After the drug-layering process, resulting pellet cores were dried for 30 min in the centrifugal coating pan so as to obtain the active ingredient-loaded pellet cores.

80 g of a polyacrylic acid resin IV was dissolved into a 720 mL of a 90 wt. % ethanol solution until the solution was clarified, so that a coating solution was obtained. 240.0 g of the active ingredient-loaded pellet cores were put into the centrifugal coating pan. The temperature in the centrifugal coating pan was regulated to be 40° C., and the air supply rate was regulated to be 70 m³*h⁻¹. Thereafter, the coating solution was pumped by the peristaltic pump into the centrifugal coating pan at the flow rate of 2 mL/min for performing the coating process, during which, the atomization pressure was controlled at 1.4 bar, and the solution supply rate was gradually increased to 6 mL/min until the coating solution was consumed. After the coating process, a temperature in the centrifugal coating pan was increased to 45° C. for fluidized drying resulting pellets for 30 min. Pellets having diameters of between 0.10 and 0.25 mm were then selected and examined to obtain qualified taste-masking pellets.

2) Preparation Process of the Orally-Disintegrating Oseltamivir Tablet

Formula for the orally-disintegrating oseltamivir tablet: 128.0 g of the taste-masking pellets, 240.0 g of mannitol as a first filler, 10.0 g of a hydroxypropyl methyl cellulose as a first adhesive, 40.0 g of a crospovidone as a disintegrant, 10.0 g of aspartame and 3.0 g of citric acid as flavoring agents, 6.0 g of magnesium stearate as a lubricant.

Preparation process: the above materials were mixed in a trough mixer for 45 min, and the mixed materials were transferred to a hopper of a tablet press machine which was configured with a die for scoring each tablet into 6 aliquots. A tablet weight and a tablet hardness were regulated to keep a tablet hardness at between 35 and 45 N, then the tableting process was performed to obtain the orally-disintegrating tablets comprising oseltamivir. The uniformity and the dissolution of each batch of the tablets were examined, and the qualified tablets were placed into a lucifugal airtight container.

It should be noted that the purified water and ethanol added in the preparation were finally evaporated from the products in the drying process.

It was known from calculation that weight percentage amounts of the components in the orally-disintegrating oseltamivir tablet were as follows: 29.3 wt. % of the taste-masking pellets, 54.9 wt. % of mannitol, 2.3 wt. % of the hydroxypropyl methyl cellulose, 9.2 wt. % of the crospovidone, 2.3 wt. % of aspartame, 0.7 wt. % of citric acid, 1.4 wt. % of magnesium stearate.

### Example 2

### Orally-Disintegrating Oseltamivir Tablet and Preparation Method Thereof

The preparation of the orally-disintegrating oseltamivir tablet of this example is the basically the same as that of Example 1 except that: the formula for the orally-disintegrating oseltamivir tablet of this example is as follows: 107.5 g of the taste-masking pellets; 176.4 g of mannitol and 25.2 g of a microcrystalline cellulose as first fillers; 8.4 g of a povidone as a first adhesive; 33.6 g of a crospovidone as a disintegrant; 8.4 g of aspartame, 2.5 g of citric acid, and 2.5 g of a strawberry essence as flavoring agents; and 5.0 g of sodium stearyl fumarate as a lubricant.

It was known from calculation that weight percentage amounts of the components in the orally-disintegrating oseltamivir tablet were as follows: 29.1 wt. % of the taste-masking pellets, 47.7 wt. % of mannitol, 6.8 wt. % of the microcrystalline cellulose, 2.3 wt. % of a povidone, 9.1 wt. % of the crospovidone, 2.3 wt. % of aspartame, 0.7 wt. % of citric acid, 0.7 wt. % of the strawberry essence, and 1.4 wt. % of sodium stearyl fumarate.

### Example 3

### Orally-Disintegrating Oseltamivir Tablet and Preparation Method Thereof

The preparation of the orally-disintegrating oseltamivir tablet of this example is the basically the same as that of Example 1 except that: the formula for the orally-disintegrating oseltamivir tablet of this example is as follows: 118.4 g of the taste-masking pellets; 350.0 g of mannitol and 70.0 g of a microcrystalline cellulose as first fillers; 16.8 g of a povidone as a first adhesive; 47.7 g of a low-substituted hydroxypropyl cellulose as a disintegrant; 17.9 g of aspartame and 5.6 g of citric acid as flavoring agents; and 8.3 g of magnesium stearate as a lubricant.

It was known from calculation that weight percentage amounts of the components in the orally-disintegrating oseltamivir tablet were as follows: 18.7 wt. % of the taste-masking pellets, 55.1 wt. % of mannitol, 11.0 wt. % of the microcrystalline cellulose, 2.6 wt. % of the povidone, 7.5 wt. % of the substituted hydroxypropyl cellulose, 2.8 wt. % of aspartame, 0.9 wt. % of citric acid, and 1.3 wt. % of magnesium stearate.

### Example 4

### Orally-Disintegrating Oseltamivir Tablet and Preparation Method Thereof

The preparation of the orally-disintegrating oseltamivir tablet of this example is the basically the same as that of Example 1 except that: the formula for the orally-disintegrating oseltamivir tablet of this example is as follows: 80.0 g of the taste-masking pellets; 150.0 g of xylitol and 150.0 g of a microcrystalline cellulose as first fillers; 8.0 g of a hydroxypropyl cellulose as a first adhesive; 30.0 g of a low-substituted hydroxypropyl cellulose as a disintegrant; 15.0 g of a stevioside as a flavoring agent; and 7.0 g of sodium stearyl fumarate as a lubricant.

It was known from calculation that weight percentage amounts of the components in the orally-disintegrating oseltamivir tablet were as follows: 18.2 wt. % of the taste-masking pellets, 34.1 wt. % of xylitol, 34.1 wt. % of the microcrystalline cellulose, 1.8 wt. % of the hydroxypropyl cellulose, 6.8 wt. % of the substituted hydroxypropyl cellulose, 3.4 wt. % of the stevioside, and 1.6 wt. % of sodium stearyl fumarate.

### Example 5

### Orally-Disintegrating Oseltamivir Tablet Phosphate and Preparation Method Thereof

Taste-masking pellets comprising oseltamivir phosphate were first prepared, mixed with other excipients according to a formula, and then tableted into tablets, so as to form the orally-disintegrating tablets comprising oseltamivir phosphate.

1) Preparation Process of the Taste-Masking Pellets Comprising Oseltamivir Phosphate

The raw material of oseltamivir phosphate and excipients were ground and screened by a 100-mesh sieve. 200.0 g of a starch and 100.0 g of oseltamivir phosphate were then put into a centrifugal coating pan. A temperature in the centrifugal coating pan was regulated to be 45° C., and an air supply rate therein was regulated to be 65 m³*h⁻¹. 300 g of a 85 wt. % ethanol solution as a second adhesive solution was sprayed by a peristaltic pump into the centrifugal coating pan at a flow rate of 3 mL/min for performing a drug-layering process, during which, an atomization pressure was controlled at 1.0 bar, and a solution supply rate was gradually increased to 6 mL/min until the second adhesive solution was consumed. After the drug-layering process, resulting pellet cores were dried for 30 min in the centrifugal coating pan so as to obtain the active ingredient-loaded pellet cores.

100 g of a polyacrylic acid resin IV was dissolved into 900 mL of a 90 wt. % ethanol solution until the solution was clarified, so that a coating solution was obtained. 240.0 g of the active ingredient-loaded pellet cores were put into the centrifugal coating pan. The temperature in the centrifugal coating pan was regulated to be 40° C., and the air supply rate was regulated to be 70 m³*h¹. Thereafter, the coating solution was pumped by the peristaltic pump into the centrifugal coating pan at the flow rate of 2 mL/min for performing the coating process, during which, the atomization pressure was controlled at 1.4 bar, and the solution supply rate was gradually increased to 6 mL/min until the coating solution was consumed. After the coating process, a temperature in the centrifugal coating pan was increased to 45° C. for fluidized drying resulting pellets for 30 min. Pellets having diameters of between 0.10 and 0.25 mm were then selected and examined to obtain qualified taste-masking pellets.

2) Preparation Process of the Orally-Disintegrating Oseltamivir Tablet Phosphate

Formula for the orally-disintegrating oseltamivir tablet phosphate: 95.0 g of the taste-masking pellets, 123.0 g of fructose and 180.0 g of xylitol as first fillers, 6.0 g of a hydroxypropyl cellulose as a first adhesive, 25.0 g of a croscarmellose sodium as a disintegrant, 2.5 g of citric acid and 2.5 g of a mint essence as flavoring agents, and 6.0 g of magnesium stearate as a lubricant.

Preparation process: the above materials were mixed in a trough mixer for 45 min, and the mixed materials were transferred to a hopper of a tablet press machine which was configured with a die for scoring each tablet into 8 aliquots. A tablet weight and a tablet hardness were regulated to keep a tablet hardness at between 35 and 45 N, then the tableting process was performed to obtain the orally-disintegrating tablets comprising oseltamivir phosphate. The uniformity and the dissolution of each batch of the tablets were examined, and the qualified tablets were placed into a lucifugal airtight container.

It should be noted that the purified water and ethanol added in the preparation were finally evaporated from the products in the drying process.

It was known from calculation that weight percentage amounts of the components in the orally-disintegrating oseltamivir tablet were as follows: 21.6 wt. % of the taste-masking pellets, 28.0 wt. % of fructose, 40.9 wt. % of xylitol, 1.4 wt. % of the hydroxypropyl cellulose, 5.7 wt. % of the croscarmellose sodium, 0.6 wt. % of citric acid, 0.6 wt. % of the mint essence, and 1.4 wt. % of magnesium stearate.

### Example 6

### Orally-Disintegrating Oseltamivir Tablet Phosphate and Preparation Method Thereof

The preparation of the orally-disintegrating oseltamivir tablet phosphate of this example is the basically the same as that of Example 5 except that: the formula for the orally-disintegrating oseltamivir tablet phosphate of this example is as follows: 97.5 g of the taste-masking pellets; 280.0 g of sucrose as a first filler; 8.6 g of a hydroxypropyl cellulose as a first adhesive; 24.4 g of a crospovidone as a disintegrant; 4.7 g of a saccharin sodium as a flavoring agent; and 5.8 g of sodium stearyl fumarate as a lubricant.

It was known from calculation that weight percentage amounts of the components in the orally-disintegrating oseltamivir tablet phosphate were as follows: 23.2 wt. % of the taste-masking pellets, 66.5 wt. % of sucrose, 2.0 wt. % of the hydroxypropyl cellulose, 5.8 wt. % of the crospovidone, 1.1 wt. % of the saccharin sodium, and 1.4 wt. % of the sodium stearyl fumarate.

### Example 7

### Orally-Disintegrating Oseltamivir Tablet Phosphate and Preparation Method Thereof

The preparation of the orally-disintegrating oseltamivir tablet phosphate of this example is the basically the same as that of Example 5 except that: the formula for the orally-disintegrating oseltamivir tablet phosphate of this example is as follows: 125.5 g of the taste-masking pellets; 365.4 g of xylitol and 36.5 g of a microcrystalline cellulose as first fillers; 15.6 g of a polyvinyl alcohol as a first adhesive; 44.5 g of a carboxymethylcellulose calcium as a disintegrant; 5.6 g of citric acid as a flavoring agent; and 9.6 g of magnesium stearate as a lubricant.

It was known from calculation that weight percentage amounts of the components in the orally-disintegrating oseltamivir tablet phosphate were as follows: 20.8 wt. % of the taste-masking pellets, 60.6 wt. % of xylitol, 6.1 wt. % of the microcrystalline cellulose, 2.6 wt. % of the polyvinyl alcohol, 7.4 wt. % of the carboxymethylcellulose calcium, 0.9 wt. % of citric acid, and 1.6 wt. % of magnesium stearate.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An orally-disintegrating oseltamivir tablet, the tablet comprising: between 10 and 50 wt. % of a taste-masking pellet, between 30 and 80 wt. % of a first filler, between 1 and 6 wt. % of a first adhesive, between 2 and 10 wt. % of a disintegrant, between 0 and 5 wt. % of a flavoring agent, between 0.5 and 2.5 wt. % of a lubricant; wherein
the taste-masking pellet comprises: an active ingredient-loaded pellet core and a coating layer;
the active ingredient of the pellet core is oseltamivir having a formula I 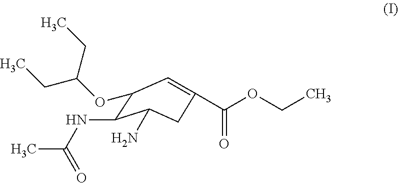 [SMILES: CCOC(=O)C1=CC(OC(CC)CC)C(NC(C)=O)C(N)C1]
or a pharmaceutically acceptable salt of oseltamivir, and accounts for between 10 and 40 wt. % of a total weight of the taste-masking pellet;
the coating layer comprises a polyacrylic acid resin IV and accounts for between 1 and 50 wt. % of the total weight of the taste-masking pellet; and
a diameter of the taste-masking pellet is between 0.10 and 0.50 mm.

2. The tablet of claim 1, wherein
the pellet core of the taste-masking pellet is prepared by a blank pellet, the drug, a second filler, a second adhesive, and an anti-sticking agent;
weight percentage amounts of the components in the pellet are as follows: between 10 and 40 wt. % of the drug, between 20 and 60 wt. % of the blank pellet, between 0 and 50 wt. % of the second filler, between 1 and 20 wt. % of the second adhesive, and between 0.5 and 5 wt. % of the anti-sticking agent;
the blank pellet is at least one selected from the group consisting of a sucrose pellet, a microcrystalline cellulose pellet, a starch pellet, a lactose-microcrystalline cellulose pellet, a starch-microcrystalline cellulose pellet, and a sucrose-starch pellet;
the second filler is at least one selected from the group consisting of sucrose, lactose, mannitol, a starch, a microcrystalline cellulose, an algal polysaccharide, a chitosan;
the second adhesive is at least one selected from the group consisting of water, ethanol, a hydroxypropyl methyl cellulose, a polyacrylic acid resin, a hydroxypropyl cellulose, a povidone, a polyvinyl alcohol, and a carboxymethylcellulose sodium; and
the anti-sticking agent is at least one selected from the group consisting of a talc, colloidal silica, magnesium stearate, calcium stearate, magnesium silicate, and glyceryl monostearate.

3. The tablet of claim 2, wherein
in the taste-masking pellet, the blank pellet is the sucrose pellet, the second filler is lactose and/or mannitol, the second adhesive is the hydroxypropyl methyl cellulose, and the anti-sticking agent is the talc; and
the weight percentage amounts of the components in the taste-masking pellet are as follows: between 10 and 30 wt. % of oseltamivir, between 5 and 50 wt. % of the polyacrylic acid resin IV, between 20 and 60 wt. % of the sucrose pellet, between 0 and 50 wt. % of lactose and/or mannitol, between 1 and 20 wt. % of the hydroxypropyl methyl cellulose, and between 0.5 and 50 wt. % of the talc.

4. The tablet of claim 3, wherein the weight percentage amounts of the components in taste-masking pellet are as follows: 23.8 wt. % of oseltamivir, 16.7 wt. % of the polyacrylic acid resin IV, 49.6 wt. % of the sucrose pellet, 7.9 wt. % of the hydroxypropyl methyl cellulose, and 2.0 wt. % of the talc.

5. The tablet of claim 1, wherein
the first filler is at least one selected from the group consisting of mannitol, xylitol, sucrose, fructose, glucose, maltose, aminoacetic acid, sorbitol, a microcrystalline cellulose, and lactose;
the first adhesive is at least one selected from the group consisting of a hydroxypropyl methyl cellulose, a polyacrylic acid resin, a hydroxypropyl cellulose, a povidone, a polyvinyl alcohol, and a carboxymethylcellulose sodium;
the disintegrant is at least one selected from a sodium carboxyl methyl starch, a crospovidone, a croscarmellose sodium, a substituted hydroxypropyl cellulose, and a carboxymethylcellulose calcium;
the flavoring agent is at least one selected from aspartame, Acesulfame-K, saccharin sodium, a dextran, a stevioside, citric acid, an essence, and a perfume; and
the lubricant is at least one selected from the group consisting of a talc, a hydrogenated vegetable oil, sodium stearyl fumarate, magnesium stearate, stearyl alcohol.

6. The tablet of claim 5, wherein
the first filler is mannitol and/or the microcrystalline cellulose; the first adhesive is the povidone; the disintegrant is the crospovidone and/or the substituted hydroxypropyl cellulose; the flavoring agent is aspartame and/or citric acid and/or a strawberry essence; and the lubricant is sodium stearyl fumarate and/or magnesium stearate.

7. The tablet of claim 6, wherein the percentage amounts of the components in the tablet are as follows:
between 10 and 50 wt. % of the taste-masking pellet having the diameter of between 0.15 and 0.35 mm, between 40 and 80 wt. % of mannitol and/or the microcrystalline cellulose, between 1 and 3 wt. % of the povidone, between 5 and 10 wt. % of the crospovidone and/or the substituted hydroxypropyl cellulose, between 1 and 4 wt. % of aspartame and/or citric acid and/or the strawberry essence, and
between 0.5 and 1.5 wt. % of sodium stearyl fumarate and/or magnesium stearate.

8. The tablet of claim 7, wherein the percentage amounts of the components in the tablet are as follows:
between 25 and 35 wt. % of the taste-masking pellet having the diameter of between 0.15 and 0.35 mm, between 40 and 50 wt. % of mannitol, between 5 and 8 wt. % of the microcrystalline cellulose, between 1 and 3 wt. % of the povidone, between 6 and 10 wt. % of the crospovidone, between 2 and 3 wt. % of aspartame, between 0.5 and 1 wt. % of citric acid, between 0.5 and 1 wt. % the strawberry essence, and between 1.0 and 1.5 wt. % of sodium stearyl fumarate.

9. The tablet of claim 1, wherein
a hardness of the tablet exceeds 40 N; the tablet is orally disintegrated within 30 seconds in the absence of bitter taste; the tablet is scored into between 4 and 12 equal parts; and an effective dose of oseltamivir is between 5 and 50 mg.

10. The tablet of claim 9, wherein the tablet is scored into between 6 and 12 equal parts; and the effective dose of oseltamivir is between 10 and 30 mg

11. A method for preparing the orally-disintegrating oseltamivir tablet of claim 1, the method comprising:
a) grinding oseltamivir, and processing the ground oseltamivir into active ingredient-loaded pellet cores using a coating machine; adding the polyacrylic acid resin IV to an ethanol solution, and coating a resulting solution on the active ingredient-loaded pellet cores using a fluidized bed or a coating pan to form a coating layer on the pellet cores, whereby yielding the taste-masking pellets; and
b) uniformly mixing the taste-masking pellets with the first filler, the first adhesive, the flavoring agent, the disintegrant, and the lubricant, and tableting a resulting mixture.
